(19) United States
(12) Patent Application Publication
(10) Pub. No.: US 2004/0002702 A1
(43) Pub. Date: Jan. 1, 2004

(54) RF device for treating the uterus

(75) Inventors: Jia Hua Xiao, Bridgewater, NJ (US); Thomas P. Ryan, Flemington, NJ (US); Alexander J. Sinton, Doylestown, PA (US)

Correspondence Address: SELITTO, BEHR & KIM, 203 MAIN STREET, METUCHEN, NJ, 08840, US

(73) Assignee: Ethicon, Inc.

(21) Appl. No.: 10/186,259

(22) Filed: Jun. 28, 2002

(51) Int. Cl. A61B 18/18

(52) U.S. Cl. 606/41

(57) ABSTRACT

An electrode instrument is provided and includes an electrode head and a pair of electrodes for emitting RF energy for use in thermal ablation therapy. The electrodes are movable between a collapsed configuration and deployed configuration. When collapsed, the electrodes are proximate to each other, and when deployed, the electrodes are spaced apart from each other. The electrode instrument includes linkage mechanisms that are used to maintain the electrodes in their deployed configuration.

### FIELD OF THE INVENTION

The present invention relates to an RF (radio frequency) device for use in the performance of thermal ablation, and more particularly, to an RF device for ablation of endometrial cells and tissue within the uterus.

### BACKGROUND OF THE INVENTION

Millions of women suffer from excessive menstrual bleeding (menorrhagia). A commonly used therapy to treat menorrhagia involves ablating the endometrial lining that is responsible for the bleeding. Such ablation has been shown to reduce the bleeding, and in some instances, to cease the menstrual bleeding.

Various methods have been used to ablate the endometrial lining of the uterus. One such method involves inserting a balloon catheter into the uterus, filling the balloon with a thermally conductive fluid, and then heating the fluid to thermally ablate the endometrial lining of the uterus. Although thermal balloon therapy is effective for treating menorrhagia in women who have a smooth uterine lining, such balloon therapy is not recommended for women who have uterine conditions such as myomas, polyps, or irregular uterine shapes.

Accordingly, there is a need for a therapy that involves the use of thermal ablation for treating menorrhagia in women who have benign uterine pathology and that is easy to use and to control.

### SUMMARY OF THE INVENTION

In accordance with the present invention, an electrode instrument is provided and used for thermal ablation therapy. An electrode head is included, which has a first and second electrode for emitting RF energy. The electrodes are movable between a collapsed configuration, in which the electrodes are proximate to each other, and a deployed configuration, in which the electrodes are spaced apart from each other. A linkage mechanism is connected between the electrodes and is used to maintain the electrodes in their deployed configuration.

Other features and aspects of the present invention will become more fully apparent from the following detailed description of the exemplary embodiments, the appended claims and the accompanying drawings.

### BRIEF DESCRIPTION OF THE DRAWINGS

For a more complete understanding of the present invention, reference is made to the following detailed description of the exemplary embodiments considered in conjunction with the accompanying drawings, in which:

FIG. 1 is a front perspective view of an RF device constructed in accordance with the present invention;

FIG. 2 is a front perspective view of an RF catheter of the RF device shown in FIG. 1, which shows an electrode head in a fully relaxed configuration;

FIGS. 3 to 5 are sequential perspective views of the RF catheter, showing the progressive movement of the electrode head as it moves from the relaxed configuration to a deployed configuration;

FIG. 6 is a view similar to the view shown in FIGS. 2 to 5, except that the electrode head is in the fully deployed configuration;

FIG. 7 is a schematic view of a female uterus and the RF device shown in FIG. 1, which shows a distal portion of the electrode head in contact with the uterine cavity;

FIG. 8 is a view similar to the view shown in FIG. 7, except that the electrode head is in the deployed configuration;

FIG. 9 is a front perspective view of an RF device in accordance with a second embodiment of the present invention, which shows a tube in a distal position;

FIG. 10 is a sequential view of the RF device of FIG. 9, showing the tube between its distal and proximal positions; and

FIG. 11 is a view similar to the view shown in FIG. 9, except that the tube is in the proximal position, fully deployed.

### DETAILED DESCRIPTION OF THE EXEMPLARY EMBODIMENT

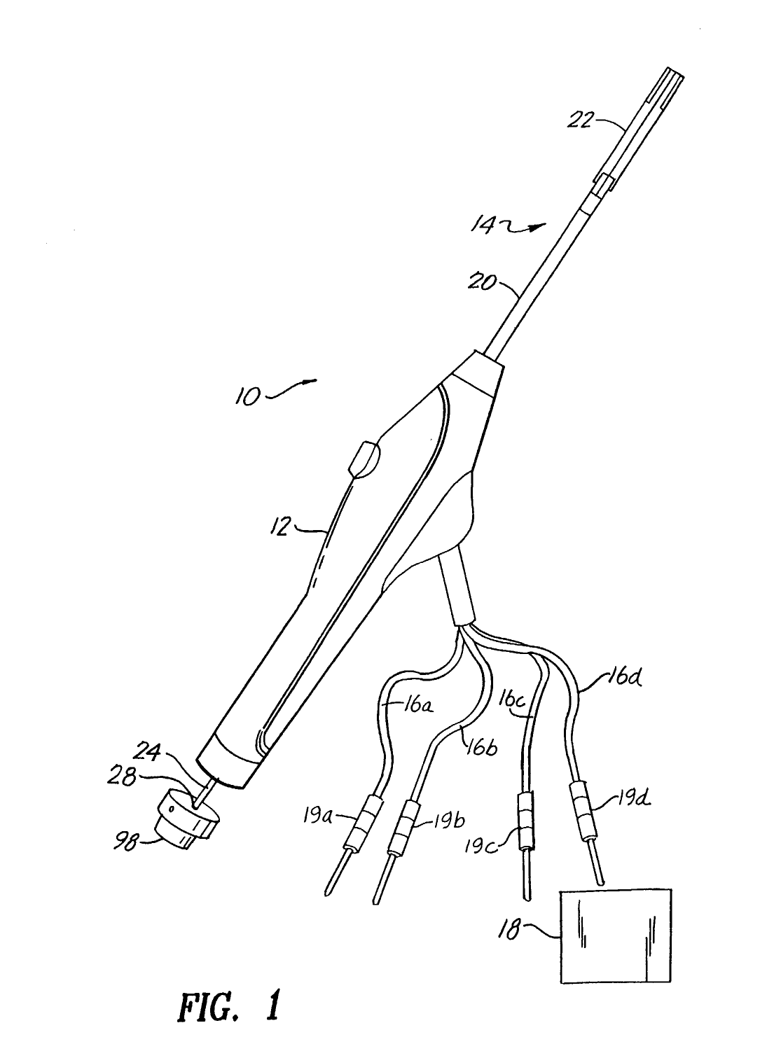

FIG. 1 shows an RF device 10 having a handgrip portion 12 and an RF catheter 14 connected thereto. Various cables 16a-d are attached to the handgrip portion 12 and are provided for connecting the RF device 10 to an RF generator 18. (shown in phantom). The RF generator 18 is electrically connected to the RF catheter 14 and serves to provide bipolar RF energy, wherein the current flow is localized between an active and return electrode, to the RF device 10. Electrical leads 19a-d are electrically connected to the RF generator 18, via one of the cables 16a-d, respectively, and extend to the RF catheter 14. During use, the electrical leads 19a-d carry RF energy from the RF generator 18 to the RF catheter 14.

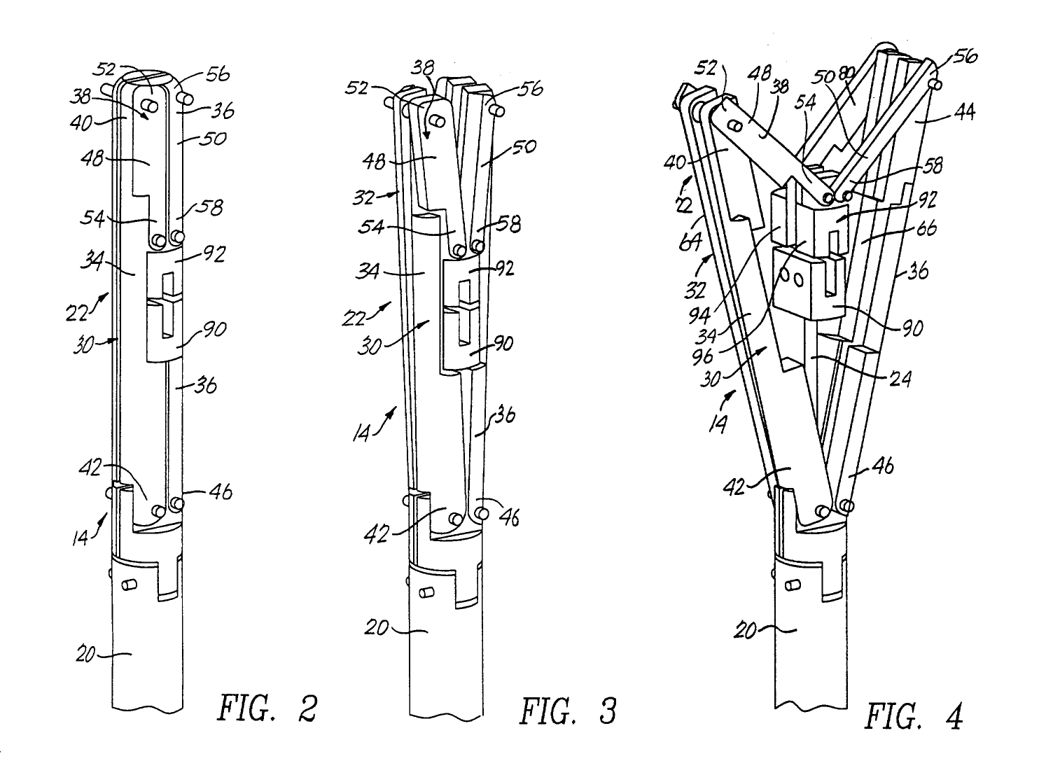

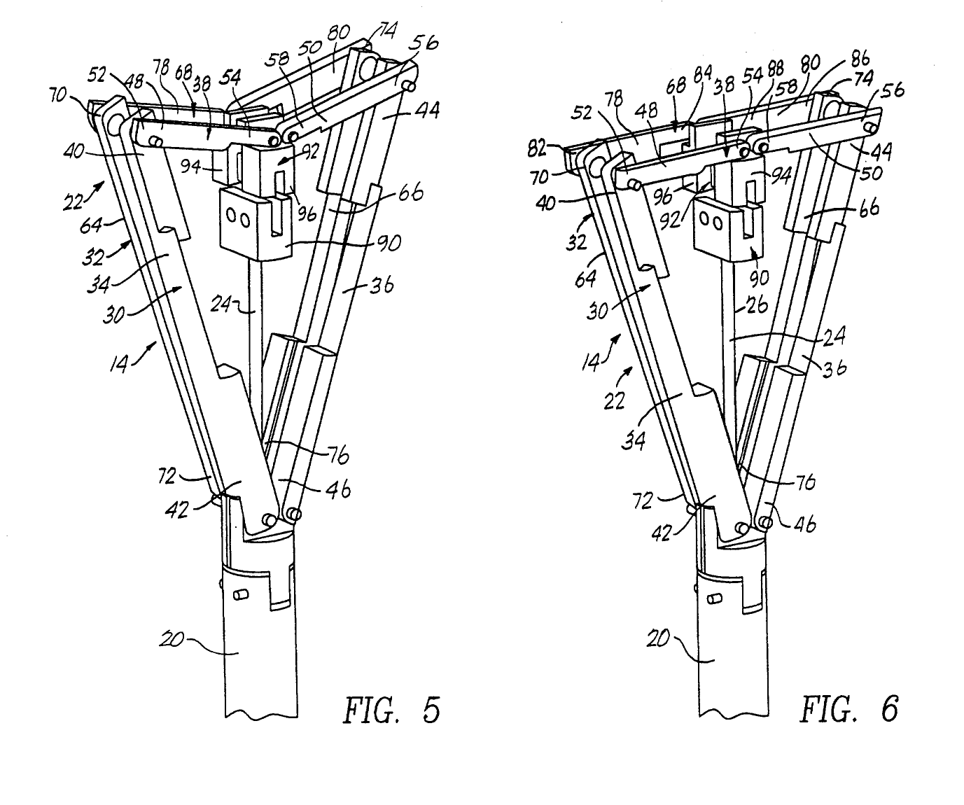

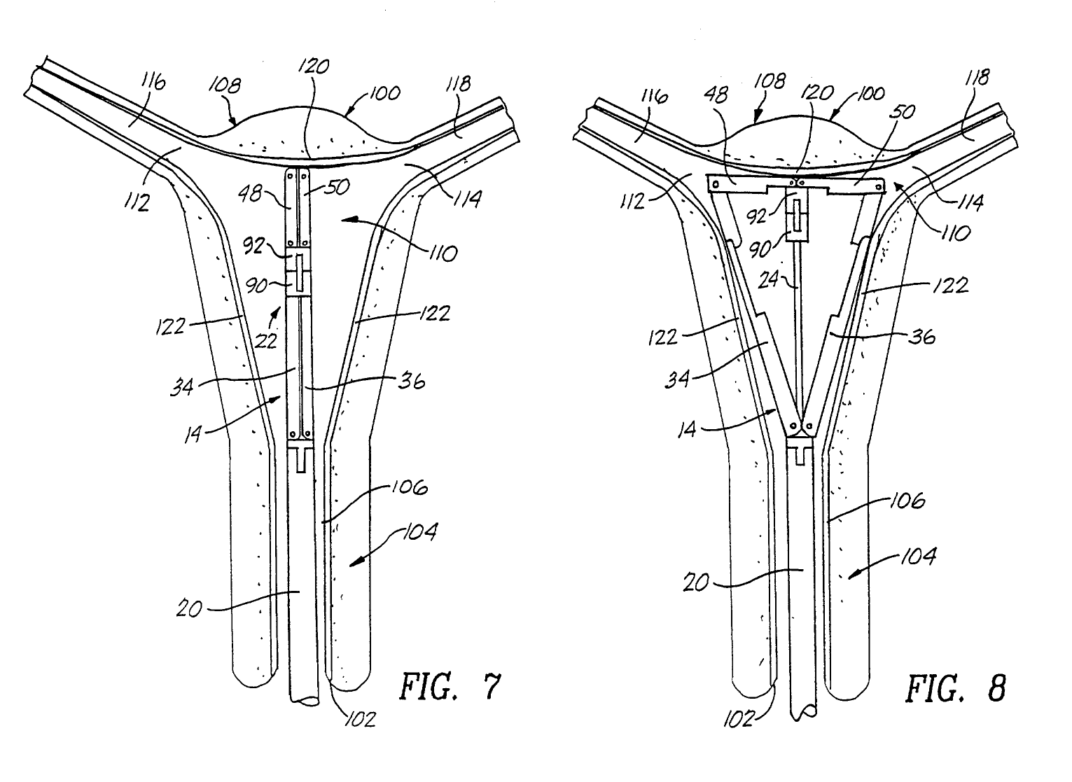

With reference to FIGS. 2 to 6, the RF catheter 14 includes an outer sheath 20 and an electrode head 22 attached thereto. For reasons to be discussed hereinafter, the electrode head 22 is sized and shaped to move between a deployed configuration as shown in FIG. 6, in which it is substantially triangularly-shaped so as to approximate the shape of the uterine cavity, and a relaxed configuration as shown in FIG. 2, in which it is substantially cylindrically-shaped so as to facilitate transcervical insertion into the uterine cavity.

Referring to FIG. 1, the RF device 10 includes an actuator rod 24 that extends between the proximal and distal ends thereof. The actuator rod 24 is sized and shaped to be received within the handgrip portion 12 and within the RF catheter 14. A distal end 26 (see FIG. 6) of the actuator rod 24 extends beyond the outer sheath 20 and into the electrode head 22. The actuator rod 24 also includes a proximal end 28 that extends beyond the handgrip portion 12.

With reference to FIGS. 2 to 6, the electrode head 22 includes a pair of RF applicators 30, 32 positioned adjacent to each other. As shown in FIG. 6, the RF applicator 30 has a pair of electrode legs, one leg includes 34 and 48, and the other leg includes 36 and 50, for delivering RF energy. The polarity of the electrode legs 34, 36 can be regulated such that electrode legs 34, 36 can be activated as an active or return electrode. The electrode leg 34 has a distal end 40 and a proximal end 42, and likewise, the electrode leg 36 has a distal end 44 and a proximal end 46. Still referring to FIG. 6, the arm 48 is electrically isolated from the arm 50, and mechanically connected to the arm 50. The arm 48 has a distal end 52 and a proximal end 54, and the arm 50 has a distal end 56 and a proximal end 58 mechanically connected, but electrically isolated, to the proximal end 54 of the arm 48. More particularly, the distal end 52 of the arm 48 is connected to the distal end 40 of the electrode leg 34 and can pivot thereabout, while the distal end 56 of the arm 50 is connected to the distal end 44 of the electrode leg 36 and can pivot thereabout. The electrode leg 34 and the arm 48 are electrically connected to each other and to the cable 16a. Also, the electrode leg 36 and the arm 50 are electrically connected to each other and to the cable 16b.

The RF applicator 30 is sized and shaped to move between a closed configuration (see FIG. 2) and an open configuration (see FIG. 6). With reference to FIG. 2, when the RF applicator 30 is in its closed configuration, the distal ends 40, 44 of the electrode legs 34, 36 are adjacent to each other, the proximal ends 42, 46 of the electrode legs 34, 36 are adjacent to each other, the distal ends 52, 56 of the arms 48, 50 are adjacent to each other, and the proximal ends 54, 58 of the arms 48, 50 are also adjacent to each other. Referring to FIG. 6, when the RF applicator 30 is in its open configuration, the distal ends 40, 44 of the electrode legs 34, 36 are spaced apart from each other so as to form a V-shaped figure, the distal ends 52, 56 of the arms 48, 50 are spaced apart from each other, and the proximal ends 54, 58 of the arms 48, 50 are adjacent to each other in a substantially linear fashion.

As shown in FIG. 6, the RF applicator 32 has a pair of electrode legs, one leg includes 64 and 78 and the other leg includes 66 and 80 for delivering RF energy. The polarity of the electrode legs 64, 66 can be regulated such that electrode legs 64, 66 can be activated as an active or return electrode. The electrode leg 64 has a distal end 70 and a proximal end 72, and likewise, the electrode leg 66 has a distal end 74 and a proximal end 76. Still referring to FIG. 6, the arm 78 is electrically isolated from the arm 80, and mechanically connected to the arm 80. The arm 78 has a distal end 82 and a proximal end 84, and the arm 80 has a distal end 86 and a proximal end 88 mechanically connected, but electrically isolated, to the proximal end 84 of the arm 78. More particularly, the distal end 82 of the arm 78 is connected to the distal end 70 of the electrode leg 64 and can pivot thereabout, while the distal end 86 of the arm 80 is connected to the distal end 74 of the electrode leg 66 and can pivot thereabout. The electrode leg 64 and the arm 78 are electrically connected to each other and to the cable 16c. Also, the electrode leg 66 and the arm 80 are electrically connected to each other and to the cable 16d.

In a bipolar fashion, the electrode leg 34 and the arm 48 can be activated as the active electrode, while the electrode leg 36 and the arm 50 can be activated as the return electrode. The electrical connections can be switched such that the electrode leg 34 and the arm 48 can be activated as the active electrode, while the electrode leg 64 and the arm 78 can be activated as the return electrode. Other electrical connections can also be switched such that the electrode leg 64 and the arm 78 can be activated as the active electrode, while the electrode leg 66 and the arm 80 can be activated as the return electrode. Further, the electrical connections can be altered such that the electrode leg 36 and the arm 50 can be activated as the active electrode, while the electrode leg 66 and the arm 80 can be activated as the return electrode. The polarity can be regulated such that any other combination of electrode legs and arms can be activated as an active electrode and as a return electrode. The polarity can then be altered at various time intervals so as to facilitate thermal coverage of the uterine cavity.

The RF applicator 32 is sized and shaped to move between a closed configuration (see FIG. 2) and an open configuration (see FIG. 6). When the RF applicator 32 is in its closed configuration, the distal ends 70, 74 of the electrode legs 64, 66 are adjacent to each other, the proximal ends 72, 76 of the electrode legs 64, 66 are adjacent to each other, the distal ends 82, 86 of the arms 78, 80 are adjacent to each other, and the proximal ends 84, 88 of the arms 78, 80 are also adjacent to each other. Referring to FIG. 6, when the RF applicator 32 is in its open configuration, the distal ends 70, 74 of the electrode legs 64, 66 are spaced apart from each other so as to form a V-shaped figure, the distal ends 82, 86 of the arms 78, 80 are spaced apart from each other, and the proximal ends 84, 88 of the arms 78, 80 are adjacent to each other in a substantially linear fashion.

Still referring to FIG. 6, a first piston 90 is attached to the distal end 26 of the actuator rod 24, while a second piston 92 is attached to the first piston 90 at one end and to the first and second links 38, 68 at an opposite end. More particularly, the second piston 92 has a first member 94 connected to the proximal ends 54, 58 of the arms 48, 50 and a second member 96 connected to the proximal ends 84, 88 of the arms 78, 80. The second piston 92 is sized and shaped to move between a first position (see FIG. 4), in which the first and second members 94, 96 are adjacent to each other such that the RF applicators 30, 32 are adjacent to each other, and a second position (see FIG. 6), in which the first and second members 94, 96 are spaced apart from each other such that the RF applicators 30, 32 move apart from each other.

As illustrated in FIGS. 2 to 6, the RF device 10 also includes a finger gripping flange 98 (see FIG. 1) connected to the proximal end 28 of the actuator rod 24, such that when the finger gripping flange 98 is pushed axially in a direction toward the electrode head 22, the actuator rod 24 slides the first and second pistons 90, 92 in a direction toward the first and second links 38, 68. As the first and second pistons 90, 92 slide toward the first and second links 38, 68, the RF applicators 30, 32 move from their closed configuration (see FIG. 2) to their open configuration (see FIG. 6). Also, as the first and second pistons 90, 92 slide toward the first and second links 38, 68, the second piston 92 moves from its first position to its second position (see FIG. 6). In the foregoing manner, the electrode head 22 is placed into its deployable configuration. When the finger gripping flange 98 is pulled axially in a direction away from the electrode head 22, the foregoing steps described above are reversed so as to place the electrode head 22 into its relaxed configuration.

In order to fully understand the advantages of the RF device 10, a brief overview of the female uterus 100 is discussed below with reference to FIGS. 7 and 8. The female uterus 100 includes an external cervical opening 102; a cervix 104 having a cervical canal 106; an uterus 108 having an uterine cavity 110; tubal ostia 112, 114; and Fallopian tubes 116, 118. The uterine cavity 110 is joined to the Fallopian tubes 116, 118 via their respective tubal ostia 112, 114. As illustrated in FIGS. 7 and 8, the uterine cavity 110 is substantially triangularly-shaped and includes a plurality of cavity walls in the form of a top wall (hereinafter referred to as a fundus 120) and side walls 122.

In operation, prior to inserting the electrode head 22 into the uterine cavity 110, the uterine sound (depth) is measured. More particularly, a sound (not shown) is inserted into the vaginal orifice (not shown) and guided through the cervical canal 106, and into the uterine cavity 110 until the sound is in contact with the fundus 120. The RF device 10 is then inserted transcervically into the vaginal orifice (not shown) until the electrode head 22 enters the cervix 104. Note that in the foregoing step, the electrode head 22 is in its fully relaxed configuration.

As illustrated in FIG. 7, the electrode head 22 is then guided in its relaxed configuration through the cervical canal 106, and into the uterine cavity 110 until the electrode head 22 is adjacent to the fundus 120 of the uterine cavity 110. Next, the electrode head 22 is deployed, in the manner described previously, into its fully deployed configuration as shown in FIG. 8, so as to contact the uterine cavity 110.

After the electrode head 22 is in its fully deployed configuration, voltage is supplied to the electrode legs 34, 36 of the RF applicator 30 and to the electrode legs 64, 66 of the RF applicator 32 such that RF energy is emitted therefrom to the tissues surrounding and between the electrode leg 34 and the arm 48, and the electrode leg 64 and the arm 78. RF energy is also emitted to the tissues surrounding and between the electrode leg 36 and the arm 50, and the electrode leg 66 and the arm 80 for a first predetermined time interval. The emitted RF energy heats and ablates these tissue areas. Then, the polarity of the electrode legs 34, 36 and of the electrode legs 64, 66 is altered such that one of the pair of electrode legs 34, 64 is activated as an active electrode and the other pair of electrode legs 36, 66 is activated as a return electrode. Voltage is supplied to the electrode legs 34, 36 of the RF applicator 30 and to the electrode legs 64, 66 of the RF applicator 32 such that RF energy is emitted therefrom to the tissues surrounding and between the electrode leg 34 and the arm 48, and the electrode leg 36 and the arm 50. RF energy is also emitted to the tissues surrounding and between the electrode leg 64 and the arm 78, and the electrode leg 66 and the arm 80 for a second predetermined time interval that is substantially equal to the first predetermined time interval. Alternatively, the first time interval does not approximate the second time interval. Then, the electrode head 22 is undeployed into its relaxed configuration. Lastly, the electrode head 22 is removed from the uterine cavity 110, the cervical canal 106, and the vaginal orifice (not shown).

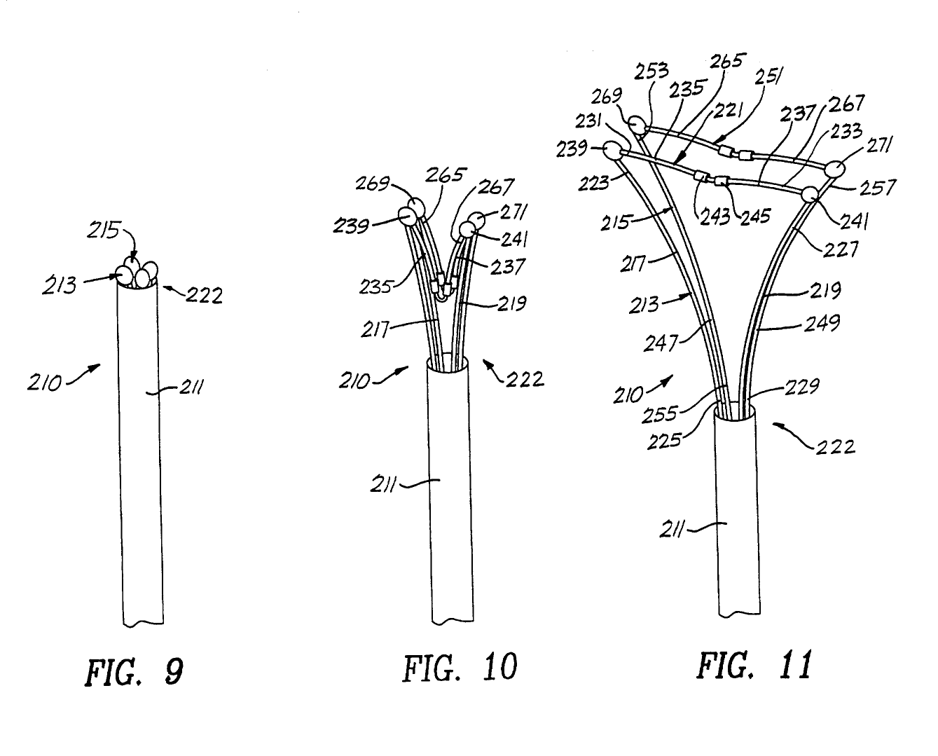

A second exemplary embodiment of the present invention is illustrated in FIGS. 9 to 11. Elements illustrated in FIGS. 9 to 11 which correspond to the elements described above with reference to FIG. 1 have been designated by corresponding reference numerals increased by two hundred. In addition, elements illustrated in FIGS. 9 to 11 which do not correspond to the elements described above with reference to FIG. 1 have been designated by odd numbered reference numerals starting with reference number 211. The embodiment of FIGS. 9 to 11 operates in the same manner as the embodiment of FIG. 1, unless it is otherwise stated.

FIG. 9 shows an RF device 210 having a tube 211 which is linearly shaped and an electrode head 222 having a pair of spring wire electrodes 213, 215 for delivering RF energy. The spring wire electrodes 213, 215 are sized and shaped to be received within the tube 211. The tube 211 is slidable between a distal position in a direction toward the spring wire electrodes 213, 215 as shown in FIG. 9, in which the spring wire electrodes 213, 215 are substantially compressed within the tube 211 so as to facilitate transcervical insertion into the uterine cavity, and a proximal position in a direction away from the spring wire electrodes 213, 215 as shown in FIG. 11, in which the spring wire electrodes 213, 215 are released from the tube 211 and assume a substantially triangularly-shape so as to approximate the shape of the uterine cavity.

The spring wire electrodes 213, 215 are resilient and have a spring or shape memory such that when released, they assume their triangular shape. It is understood that the spring wire electrodes 213, 215 can be made from any spring or shape memory material.

With reference to FIG. 11, the spring wire electrode 213 is a continuous wire that forms a three-sided figure when released. More particularly, the spring wire electrode 213 forms a pair of sides, one side includes 215 and 235, and the other side includes 217 and 237, for delivering RF energy. The side 217 has a distal end 223 and a proximal end 225, and likewise, the side 219 has a distal end 227 and a proximal end 229. Still referring to FIG. 11, the central link 221 includes a first end 231 connected to the distal end 223 of the side 217 and can pivot thereabout and a second end 233 connected to the distal end 227 of the side 219 and can pivot thereabout. The central link 221 is bendable such that when the tube 213 is in its distal position, the central link 221 bends so as to form a pair of substantially equal-sized segments 235, 237 (see FIG. 10), which facilitates insertion into the tube 213. The segment 235 is electrically isolated from the segment 237, and mechanically connected to the segment 237.

A first spherical ball 239 covers the distal end 223 of the side 217 and the first end 231 of the central link 221, while a second spherical ball 241 covers the distal end 227 of the side 219 and the second end 233 of the central link 221. The spherical ball 239 is used to electrically connect the side 217 to the segment 235. Likewise, the spherical ball 241 is used to electrically connect the side 219 to the segment 237. The balls 239, 241 are spherically shaped so as to provide a smooth surface for contact with the uterus. A pair of tabs 243, 245 is included and covers the central link 221. The tabs 243, 245 are used for electrical insulation and to mechanically connect the segment 235 to the segment 237.

With reference to FIG. 11, the spring wire electrode 215 is also a continuous wire that forms a three-sided figure when released. More particularly, the spring wire electrode 215 forms a pair of sides, one side includes 247 and 265 and the other side includes 249 and 267. The side 247 has a distal end 253 and a proximal end 255, and likewise, the side 249 has a distal end 257 and a proximal end 259. Still referring to FIG. 11, the central link 251 has a third end 261 connected to the distal end 253 of the side 247 and can pivot thereabout and a fourth end 263 connected to the distal end 257 of the side 249 and can pivot thereabout. The central link 251 is bendable such that when the tube 243 is in its distal position, the central link 251 bends so as to form a pair of substantially equal-sized segments 265, 267 (see FIG. 10), which facilitates insertion into the tube 213. The segment 265 is electrically isolated from the segment 267, and mechanically connected to the segment 267.

A third spherical ball 269 covers the distal end 253 of the side 247 and the first end 261 of the central link 251, while a fourth spherical ball 271 covers the distal end 257 of the side 249 and the second end 263 of the central link 251. The spherical ball 269 is used to electrically connect the side 247 to the segment 265. Likewise, the spherical ball 271 is used to electrically connect the side 249 to the segment 267. The balls 269, 271 are spherically shaped so as to provide a smooth surface for contact with the uterus. A pair of tabs 273, 275 is included and covers the central link 251. The tabs 273, 275 are used for electrical insulation and to mechanically connect the segment 265 to the segment 267. The polarity can be regulated such that any combination of the sides and the segments can be activated as an active electrode and as a return electrode.

It can be appreciated that the present invention provides numerous advantages. For instance, the present invention enables the use of thermal ablation therapy for treating menorrhagia in women, even if they have benign uterine pathology, without employing a balloon in the uterine cavity 110 (see FIG. 7). The RF device 10, 210 can treat menorrhagia without requiring surgery and can be used in a physician's office. The RF device 10, 210 can penetrate deeply into the uterine cavity 110 such that a high amenorrhea rate (no bleeding) is achieved. Because the electrode legs 34, 36, 64, 66 and the arms 48, 50, 78, 80 can be connected in various bipolar arrangements and switches electrical connections at various time intervals, increased thermal coverage is achieved.

It should be noted that the RF device 10, 210 can have numerous modifications and variations. For instance, the RF device 10, 210 can be either non-disposable or disposable. The RF device 10, 210 can be either bipolar as described previously, monopolar with a return or ground electrode placed on the body, or sesquipolar. Although a pair of RF applicators 30, 32 of the RF electrode 10 is shown, the number of RF applicators 30, 32 can vary. Likewise, although a pair of spring wire electrodes 213, 215 is shown, the number of spring wire electrodes 213, 215 can vary. In one aspect, the RF applicators 30, 32 are formed as a single piece (not shown), which may be suitable for delivering RF energy in monopolar fashion. Additional electrode legs can be employed within the RF device 10. In one aspect, a third electrode leg (not shown) is provided between the electrode legs 34, 36. The electrode legs 34, 36 can be segmented so as to regulate the amount of RF energy emitted from the RF device 10. The RF devices 10, 210 can be operated in water, dextrose, or sorbitol as non-conductive and non-ionic solutions. Further, the RF device 10, 210 can include flexible members (not shown) to improve conformity to the uterine cavity 110. In one aspect, the tube 211 is stationary and the spring wire electrodes 213, 215 move relative to the tube 211 by using an actuating mechanism (not shown). The first and second links 38, 68 of the RF device 10 and the central links 221, 251 of the RF device 210 may be conductive or non-conductive. All such variations and modifications are intended to be included within the scope of the invention as defined in the appended claims.

What is claimed is:

1. An electrode instrument used for thermal ablation therapy, comprising an electrode head having at least a first and second electrode for emitting RF energy, said first and second electrodes being movable between a collapsed configuration, in which said first and second electrodes are proximate to each other, and a deployed configuration, in which said first and second electrodes are remote from each other and a first linkage mechanism connected between said first and second electrodes, for maintaining said first and second electrodes in their deployed configuration.

2. The electrode instrument of claim 1, further comprising an actuator rod connected to said first linkage mechanism, said actuator rod cooperating with said first linkage mechanism to move said first and second electrodes between their collapsed and deployed configurations.

3. The electrode instrument of claim 2, wherein said first linkage mechanism is movable between a first position, in which said first linkage mechanism is substantially parallel to said first and second electrodes when said first and second electrodes are in their collapsed configuration, and a second position, in which said linkage mechanism is at an acute angle relative to said first and second electrodes when said first and second electrodes are in their deployed configuration.

4. The electrode instrument of claim 3, wherein said first linkage mechanism includes a first arm electrically connected to said first electrode and a second arm connected to said first arm, said second arm being electrically connected to said second electrode.

5. The electrode instrument of claim 4, wherein said electrode head assumes a substantially cylindrical shape when said first and second electrodes are in their said collapsed configuration, and wherein said electrode head assumes a substantially triangular shape when said first and second electrodes are in their said deployed configuration.

6. The electrode instrument of claim 5, wherein one of said first and second electrodes is an active electrode and the other of said first and second electrodes is a return electrode.

7. The electrode instrument of claim 6, wherein said electrode head further includes a third and fourth electrode for emitting RF energy, said third and fourth electrodes being movable between a collapsed configuration, in which said third and fourth electrodes are proximate to each other, and a deployed configuration, in which said third and fourth electrodes are remote from each other, and a second linkage mechanism connected between said third and fourth electrodes, for maintaining said third and fourth electrodes in their deployed configuration.

8. The electrode instrument of claim 7, wherein said third electrode is positioned adjacent said first electrode and said fourth electrode is positioned adjacent to said second electrode.

9. The electrode instrument of claim 8, wherein the polarity of said first, second, third, and fourth electrodes can be selectively varied.

10. The electrode instrument of claim 9, wherein one of said first, second, third, and fourth electrodes can be an active electrode for a first predetermined time period and can be a return electrode for a second predetermined time period.

11. The electrode instrument of claim 10, wherein said first and third electrodes emit RF energy between each other, and said second and fourth electrodes emit RF energy between each other.

12. The electrode instrument of claim 11, wherein said first and second electrodes emit RF energy between each other, and said third and fourth electrodes emit RF energy between each other.

13. The electrode instrument of claim 12, wherein said first, second, third, and fourth electrodes are sized and shaped to emit bipolar RF energy.

14. The electrode instrument of claim 1, wherein said first and second electrodes are sized and shaped to emit monopolar RF energy.

15. The electrode instrument of claim 1, wherein said electrode head further includes a tube sized and shaped so as to receive said first and second electrodes when said first and second electrodes are in their said collapsed configuration.

16. The electrode instrument of claim 15, wherein said first and second electrodes have spring memory characteristics.

17. The electrode instrument of claim 16, wherein said tube is moveable between a distal position, in which said first and second electrodes are substantially compressed within said tube, and a proximal position, in which said first and second electrodes are extended from said tube.

18. The electrode instrument of claim 17, wherein said first and second electrodes have a predetermined shape set therein.

19. The electrode instrument of claim 18, wherein said first and second electrodes assume a substantially triangular shape when said tube is in its said proximal position.

20. An electrode instrument used for thermal ablation therapy, comprising an electrode head having at least a first and second electrode, said first and second electrodes being movable between a collapsed configuration, in which said first and second electrodes are proximate to each other, and a deployed configuration, in which said first and second electrodes are remote from each other, and a linkage mechanism connected between said first and second electrodes; and an actuator rod connected to said linkage mechanism so as to cooperate with said linkage mechanism to move said first and second electrodes between their collapsed and deployed configurations.
